(19) United States
(12) Patent Application Publication
(10) Pub. No.: US 2026/0021238 A1
(43) Pub. Date: Jan. 22, 2026

(54) DRAINAGE DEVICES WITH POROUS PLUGS

(71) Applicants: YEDA RESEARCH AND DEVELOPMENT CO. LTD., Rehovot (IL); HEALTH CORPORATION NEXT TO SHAMIR MEDICAL CENTER (ASSAF HAROFEH) (R.A.), Zerifin (IL)

(72) Inventors: Brian BERKOWITZ, Rehovot (IL); Lior HELLER, Zerifin (IL)

(73) Assignees: YEDA RESEARCH AND DEVELOPMENT CO. LTD., Rehovot (IL); HEALTH CORPORATION NEXT TO SHAMIR MEDICAL CENTER (ASSAF HAROFEH) (R.A.), Zerifin (IL)

(21) Appl. No.: 19/340,758

(22) Filed: Sep. 25, 2025

Related U.S. Application Data
(63) Continuation of application No. PCT/IL2024/050335, filed on Apr. 2, 2024.

(30) Foreign Application Priority Data
Apr. 3, 2023 (IL) 301916

(51) Int. Cl. A61M 1/00

(52) U.S. Cl. CPC A61M 1/84; A61M 1/79; A61M 2202/0014; A61M 2202/0405; A61M 2205/0216

(57) ABSTRACT

The present disclosure relates to drainage devices designed to enable drainage of bodily fluids while reducing the risk of drainage openings being clogged by tissues surrounding the device. In one example, a drainage device includes a drainage tube and a porous plug attached to a distal portion of the drainage tube. The porous plug defines a plurality of pores that includes interconnected channels fluidly connecting between pore outer openings along an exposed outer surface of the plug, and pore inner openings along an inner uncovered surface of the plug, such that upon application of suction to the drainage tube, bodily fluid can flow, through the pore outer openings, pore interconnected channels, and pore inner openings, towards a lumen of the drainage tube.

### CROSS-REFERENCE TO RELATED APPLICATIONS

This application is a continuation of International Application No. PCT/IL2024/050335, filed Apr. 2, 2024, which claims the benefit of Israel Application No. 301916, filed Apr. 3, 2023, each of which is expressly incorporated herein by reference thereto.

### FIELD

The present disclosure relates to drainage devices configured to drain bodily fluids from a patient's tissue, such as an organ or a limb.

### BACKGROUND

Patients with lymphedema, otherwise known as “Lymphoedema” or “lymphatic obstruction”, experience an accumulation of fluid in their limbs. This is caused by a compromised lymphatic system, which leads to swelling in the arms and/or legs as well as other parts of the body. The lymphatic system is responsible for collecting and filtering the body's interstitial fluid. Primary Lymphedema can occur if the lymphatic vessels are impaired or missing, with onset either at birth, puberty (praecox), or adulthood (tarda). Secondary Lymphedema is caused by damage to the lymph vessels or removal of lymph nodes due to cancer treatment including surgery and/or radiation therapy. This type of Lymphedema can also be seen in the lower limbs or groin after surgery for ovarian, uterine, or colon cancer, which requires lymph node removal.

Though a cure is not available, treatments may help to improve outcomes. These treatments commonly include compression therapy, good skin care, exercise, and manual lymphatic drainage (MLD), together known as combined decongestive therapy.

In some cases, active drainage of lymphatic fluids is required. However, existing catheters, inserted into the muscular and fatty tissues of the swollen limbs, often become clogged by surrounding tissues. Thus, there is a need for improved devices that can drain bodily fluids from edematous tissue, while mitigating the risk of clogging.

### SUMMARY

One of the major obstacles in draining bodily fluids from organs, tissues or cavities, in cases such as lymphedema is that the drainage catheters, and specifically the catheter openings, are being clogged by tissue (e.g., fat tissue or muscle tissue), blood, or other non-dissolved components, when a negative pressure is applied to drain the fluids. The present disclosure is directed towards drainage devices for evacuating excessive bodily fluids from an organ in the subject's body, while mitigating the risk of drainage openings being clogged by surrounding tissues.

In one of its basic configurations, a sterile drainage device for draining bodily fluids comprises a drainage tube and a porous plug. This basic configuration can preferably be provided with any one or more of the features described elsewhere herein, in particular with those of the examples described hereafter. However, it should be understood that the basic configuration can preferably also be provided with any one or more of the features shown in the figures and/or described in conjunction with the figures, either in addition to or alternatively to the features of the examples described hereafter.

In some examples, the drainage tube comprises a tube distal portion, and defines a tube lumen extending along a central longitudinal axis.

In some examples, the porous plug comprises a plug proximal portion attached to the tube distal portion, a plug exposed outer surface, a plug inner uncovered surface, and a plurality of pores.

In some examples, the plug exposed outer surface is facing away from the central longitudinal axis.

In some examples, the plug inner uncovered surface is exposed to, and is in fluid communication with, the tube lumen.

In some examples, the pores comprise a plurality of pore outer openings at the plug exposed outer surface, a plurality of pore inner openings at the plug inner uncovered surface, and a plurality of pore interconnected channels extending through the porous plug.

In some examples, the pore interconnected channels extend between the pore outer openings and the pore inner openings, such that the pore outer openings are in fluid communication, via the pore interconnected channels and the pore inner openings, with the tube lumen.

In some examples, maximal bendability and maximal compressibility values are tested by bendability and compressibility test performed on a porous rectangular cuboid sample.

In some examples, the sample is made from the same material of the porous plug.

In some examples, the sample comprises a plurality of pores having the same dimensions and spatial arrangement as the pores of the porous plug.

In some examples, the sample has a length×width×height of 50×10× 6 mm.

In some examples, the sample comprises a lumen extending from a first end of the sample and terminating proximal to a second end of the sample.

In some examples, the lumen of the sample has a length of 45 mm.

In some examples, the lumen of the sample has a diameter of 1.5 mm.

In some examples, the sample is not bendable by more than 10^{c} when a bending force of at least 10N is applied to the second end of the sample, while the first end of the sample is affixed in position during the bendability test.

In some examples, the sample is not compressible by more than 10% when a compressive force of at least 10N is applied to a cross-section thereof during the compressibility test.

In some examples, the porous plug is close-ended at a plug distal portion thereof, such that fluid communication between the tube lumen and an environment surrounding the porous plug is achieved solely through the plurality of pores.

In some examples, the porous plug can comprise a first surface extending between two lateral sides of the porous plug, and a second surface extending between the two lateral sides, wherein the first surface can optionally be concave relative to the central longitudinal axis, wherein the second surface can optionally be convex relative to the central longitudinal axis.

In some examples, the sterile drainage device can comprise an access port attached to a proximal portion of the drainage tube.

In some examples, the access port can comprise a housing defining a chamber which is in fluid communication with the tube lumen, and a self-sealing septum attached to the housing and enclosing the chamber.

In some implementations, the sterile drainage device can comprise an implantable pump attached to a proximal portion of the drainage tube, and an outlet tube attached to the implantable pump, the outlet tube optionally extending from the implantable pump to an outlet opening of the outlet tube.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIG. 1 shows an exemplary drainage device partially implanted in a patient's body.

FIG. 2A shows a side view in perspective of a distal portion of an exemplary drainage device.

FIG. 2B shows a cross-sectional view of the drainage device of FIG. 2A.

FIG. 2C shows an enlarged cross-sectional view of a portion of the porous plug of the drainage device of FIG. 2B.

FIG. 3 shows a cross-sectional view of an exemplary drainage device having its drainage tube inserted into a full-body porous plug.

FIG. 4 shows a cross-sectional view of an exemplary drainage device having its drainage tube inserted into a cup-shaped hollow porous plug.

FIG. 5A shows a view in perspective of an exemplary hip-flask shaped porous plug.

FIG. 5B shows view in perspective of an exemplary drainage device comprising the porous plug of FIG. 5A.

FIG. 5C shows a cross-sectional view of the drainage device of FIG. 5B.

FIGS. 5D-F show cross-sectional views of exemplary drainage devices with various plug lumen shapes.

FIG. 6A shows an exemplary drainage device completely implanted in a patient's body, the drainage device comprising an implantable access port attached to the drainage tube.

FIG. 6B shows an enlarged sectional view of a proximal portion of the drainage device of FIG. 6A.

FIG. 6C shows an exemplary drainage device comprising an extracorporeal access port.

FIG. 7 shows an exemplary drainage device completely implanted in a patient's body, the drainage device comprising an internal pump attached to the drainage tube and to an outlet tube.

FIGS. 8A and 8B show exemplary threaded barbed connectors.

FIG. 9 shows a view in perspective of an exemplary hip-flask shaped porous plug with an internal thread at the plug proximal end.

FIG. 10 shows a cross-sectional view of the drainage device with the porous plug of FIG. 9 attached to a drainage tube via a threaded barbed connector.

FIG. 11A shows an exemplary rectangular cuboid porous sample that can be used for testing bendability of a porous plug. FIG. 11C shows a schematical side view of such a rectangular cuboid.

FIG. 11B shows a schematical side view of a rectangular cuboid porous sample subjected to a bending force F_{B} applied to a free end thereof.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a,” “an,” and “the” include the plural forms unless the context clearly dictates otherwise. Additionally, the terms “have” or “includes” means “comprises”. Further, the terms “engaged”, “connected”, “coupled”, and “attached”, as used herein, are interchangeable and generally mean physically and/or mechanically coupled or linked and does not exclude the presence of intermediate elements between the coupled or associated items absent specific contrary language. As used herein, “and/or” means “and” or “or”, as well as “and” and “or”.

As used herein, the term “about”, when referring to a measurable value such as an amount, a temporal duration, and the like, is meant to encompass variations of +/−10%, or +/−5%, +/−1%, or even +/−0.1% from the specified value.

The term “at least” when referring to a numerical value has the meaning of “equal to or more”.

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (e.g., inner and outer, upper and lower, above and below, left and right, and proximal and distal) may be used to facilitate discussion of the drawings and principles herein but are not intended to be limiting.

It should be understood that the disclosed examples can be adapted to drain fluid from an organ or a limb of a patient's body (as well as any internal cavity or space in a patient's body) to an external bag or other receptacle. Alternatively, the disclosed examples can be adapted to drain bodily fluids from one location in the subject's body, such as limb, to another location, such as abdomen.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

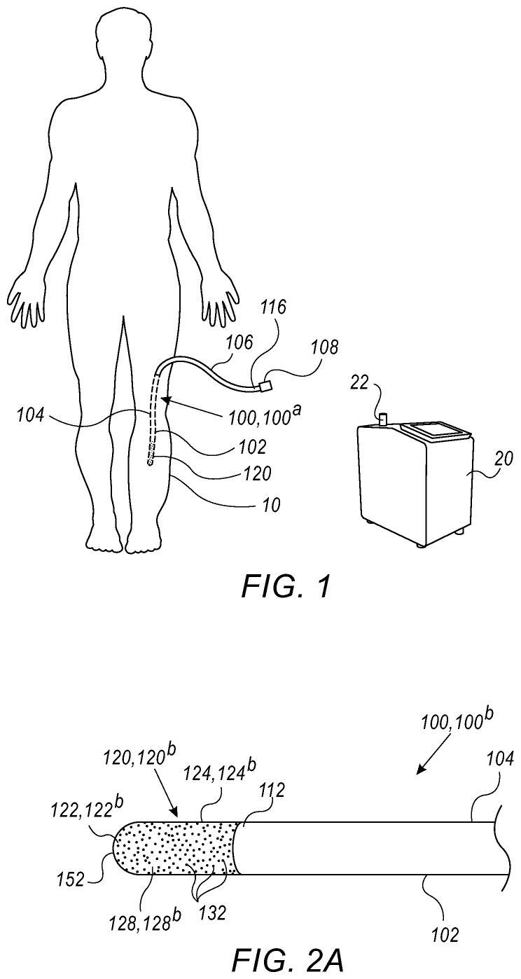

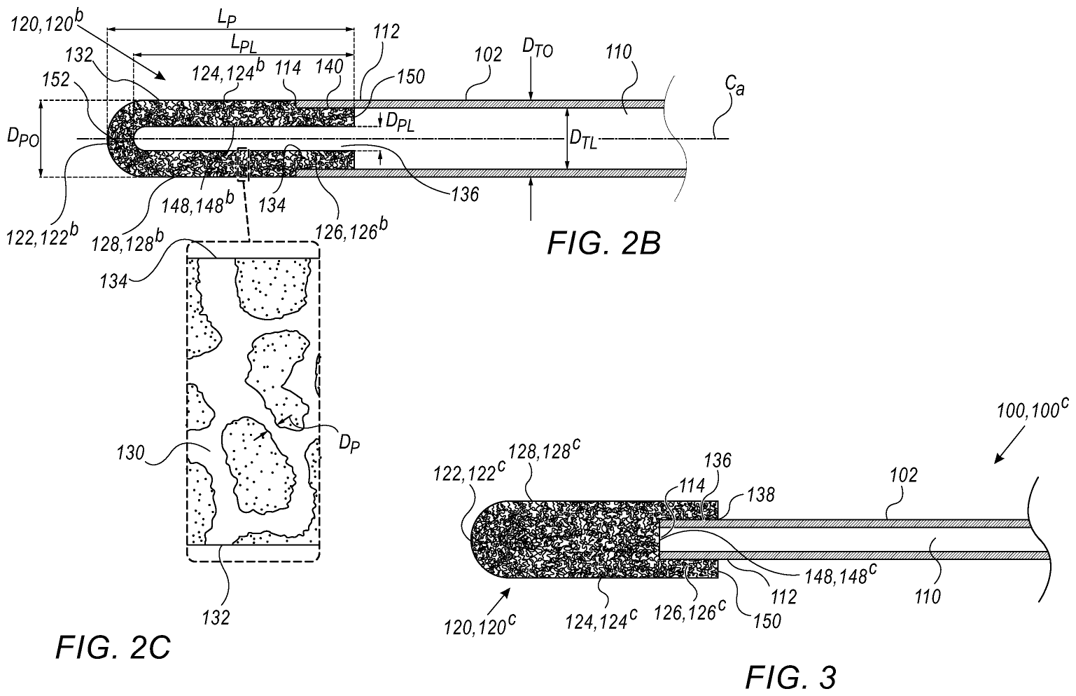

FIG. 1 illustrates an exemplary drainage device 100 at least partially implanted in an organ of a patient's body, such as a swollen limb (or any other tissue, organ, cavity) 10, and configured to drain excess fluids therefrom. The drainage device comprises a drainage tube 102 extending from a tube distal portion 112 (as shown in FIG. 2A) to a tube proximal portion 116, and a porous plug 120 attached to the distal portion 112 of the drainage tube 102.

The term “organ”, as used herein, refers to an organ in a patient's body from which the bodily fluids may be transferred, via catheter and/or tube lumens for example to an external location. In some examples, the organ is a leg or a hand. Non-limiting examples of bodily fluid are lymph fluid, blood, and interstitial fluid.

The terms “catheter” and “tube”, as used herein, refer to a thin, flexible tube or cannula that can be extruded from medical grade materials, including an outer wall which is flexible at least in a portion if not over most if not all of its length, and a lumen extending from one end to another end of the catheter or tube, such as between a proximal and a distal end thereof.

As used herein, the terms “proximal” and “distal” refer to the direction closer to and away from, respectively, a practitioner who would implant or insert the drainage device 100. Thus, for example, the end of the drainage device 100 containing a porous plug 120 would be the distal end, while the opposite end would be the proximal end.

FIGS. 2A and 2B show a side view and a cross-sectional view, respectively, of a distal portion of an exemplary drainage device 100. FIG. 2C shows an enlarged cross-sectional view of a portion of the porous plug 120 of FIG. 2B. The drainage tube 102 defines a tube lumen 110 extending along central longitudinal axis Cₐ, the tube lumen 110 terminating at a tube distal opening 114. A porous plug 120 extends along a plug total length L_{P} between a plug proximal end 150 and a plug distal end 152. The porous plug 120 includes a plug distal portion 122 extending proximally from the plug distal end 152, a plug proximal portion 126 extending distally from the plug proximal end 150, and a plug medial portion 124 extending between the plug proximal portion 126 and the plug distal portion 122. In some examples, the plug medial portion 124 can be cylindrically shaped and define a plug outer diameter D_{PO} of the porous plug 120. The drainage tube 102 defines a tube outer diameter D_{TO}, and the tube lumen 110 defines a tube lumen diameter D_{TL}. In some examples, the tube distal portion 112 can be attached to the plug proximal portion 126.

The porous plug 120 has a plug exposed outer surface 128, defined as the surface of the porous plug 120 which faces and is exposed to the environment surrounding the porous plug 120. In some examples, the exposed outer surface 128 comprises an outer surface defined by at least the plug distal portion 122 and the plug medial portion 124. The porous plug 120 also has a plug inner uncovered surface 148, defined as the surface of the porous plug 120 which is exposed to and oriented, directly or via a cavity of the porous plug, towards the tube lumen 110.

Porous plug 120 further comprises a plurality of pore interconnected channels 130 extending between a plurality of pore outer openings 132 at the exposed outer surface 128 and a plurality of pore inner openings 134 at the plug inner uncovered surface 148. The pore interconnected channels 130 are a network of interconnected passageways extending through the porous plug 120, such that the plurality of pore outer openings 132 are in fluid communication with the plurality of pore inner openings 134 via the pore interconnected channels 130. Since the pore inner openings 134 defined at the plug inner uncovered surface 148 are exposed to, and thus are in fluid communication with, the tube lumen 110, the pore outer openings 132 at the exposed outer surface 128 are in fluid communication with the tube lumen 110 as well.

The porous plug 120 can optionally be close-ended at its distal portion 122, meaning that the plug distal portion 122 does not include any opening that is larger in size than the pore outer opening 132. The porous plug 120 may have any non-traumatic shape such as a cylinder, cuboid, pyramid, conical, spherical, curbed structure or a cup. In some examples, the distal end portion 122 is shaped as an atraumatic distal end portion 122, such as by being curved without having any sharp edges. Considering the non-traumatic shape in such implementations, the shape of the porous plug 120, such as at least along the plug exposed outer surface 128, can be relatively smooth (i.e., without defining any sharp edges or corners facing the circumferential or distal directions, while still including, for example, pore outer openings 132 defined thereon).

The term “porous plug” refers to a close-ended plug comprising a plurality of pores which are implemented in the form of a plurality of pore interconnected channels 130 that form a cave-shape structure between pore outer openings 132 and pore inner opening 134. The term “pore opening”, as used herein with respect to any of pore outer openings 132 and pore inner openings 134, refers to an opening which may include a hole, tear, cavity, aperture, break, gap, or perforation on the surface of the plug, such as any of the plug exposed outer surface 128 and the plug inner uncovered surface 148. The term “pores”, as used herein, refers to the combined structure of the pore outer openings 132, pore inner opening 134, and pore interconnected channels 130 extending therebetween. The pore openings 132, 134 are interconnected via a network of internal channels 130 to form a fluid communication between the plug exposed outer surface 128 and the plug inner uncovered surface 148. Typically, the pore interconnected channels 130 channels are twisty and not linear and are predefined by features of the material from which the plug is prepared.

As shown in FIG. 2C, the porous plug 120 defines a pore size D_{P}, which is the size of the space between solid surfaced defined around the pores of porous plug 120. Given that the pore space consists of an irregular network of pores, the pore size can vary at different region of the pores, such as being narrower at pore throats (D_{P} is indicated at a region of such a pore throat in FIG. 2C) and relatively larger at wider body portions of the pore interconnected channels 130. Pore size D_{P} is defined at any portion of the pores, including the pore interconnected channels 130, the pore outer openings 132, and the pore inner opening 134. The term “pore size”, as used herein, refers to a dimension of a pore, such as a dimension of a pore outer opening, a dimension of a pore inner opening, or a dimension of a cross-section (along a plane parallel to the central longitudinal axis Cₐ) of a pore interconnected channel. In some examples, the pore size can refer to the longest dimension of a pore, e.g., a diameter of a pore having a circular cross section, or the length of the longest cross-sectional chord that can be constructed across a pore having a non-circular cross-section. In some examples, the pore size can refer to the shortest dimension of a pore.

A mean pore size D_{Pm} can be defined as a mean or averaged value of the pore sizes D_{P}. In some examples, the mean pore size D_{Pm} is from about 1 to about 60 micrometers (μm). In some examples, the mean pore size is from about 1 to about 50 μm. In some examples, the mean pore size is from about 1 to about 20 μm. In some examples, the mean pore size D_{Pm} is from about 2 to about 15 μm, from about 3 to about 12 μm, from about 4 to about 10 μm, from about 5 to about 8 μm, from about 5 to about 7 μm, from about 4 to about 8 μm, or about 6 μm. In some examples, the mean pore size D_{Pm} of the interconnected channels 130 is equal to the mean pore size of pore openings 132 and/or 134.

A maximal pore size D_{PMX} can be defined as a maximal value of the pore sizes D_{P}. A maximal pore size D_{Px} can be defined as the maximal size along any portion of the pores, including any of the pore outer openings 132, pore inner openings 134, and pore interconnected channels 130. An outer maximal pore size can be similarly defined as the maximal size of the pore outer openings 132, without referring to the pore interconnected channels for example. According to some examples, the outer maximal pore size is not greater than 60 μm. In some examples, the outer maximal pore size is not greater than 50 μm. According to some examples, the outer maximal pore size is less than 60 μm. According to some examples, the outer maximal pore size is from 1 to 60 μm.

The pore size (including any of a mean pore size and/or maximal pore size) and porosity of a porous plug 120 can be measured by standardized techniques, such as mercury porosimetry, capillary flow porometry, and nitrogen adsorption. When a plug 120 includes a plug proximal opening 138 at its proximal end 150, it is to be understood that the plug proximal opening 138 is not considered a pore for that matter. In some examples, the maximal pore size D_{PMX} is from about 1 to about 60 μm, or from about 20 to about 50 μm. In some examples, the maximal pore size D_{PMX} is not greater than 100 μm. In some examples, the maximal pore size D_{PMX} is less than 100 μm, and optionally greater than 1 μm.

As used herein, the terms “porous” and “porosity” are generally used to describe a structure having a connected network of pores or void spaces (which can, for example, be openings, interstitial spaces or other channels) throughout its volume. The term “porosity” is a measure of void spaces in a material, and is a fraction of volume of voids over the total volume, as a percentage between 0 and 50% (or between 0 and 0.5).

The mean pore size may be measured by any known method. For example, the pore size and porosity can be measured by standardized techniques, such as mercury porosimetry, capillary flow porometry, and nitrogen adsorption.

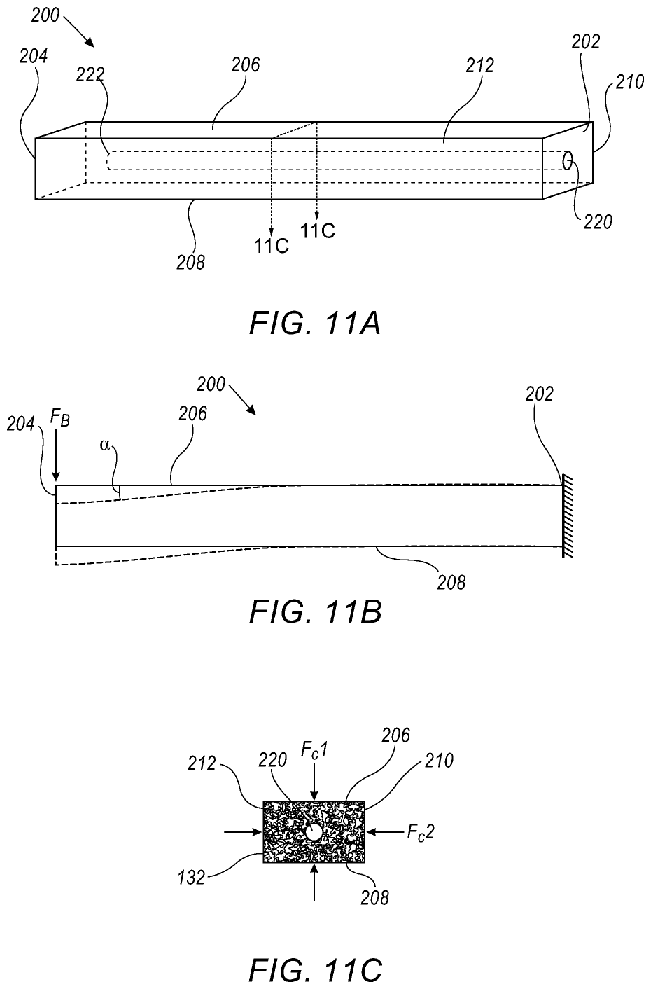

In some examples, the porous plug 120 is substantially non-bendable and substantially non-squeezable, which can facilitate easier insertion thereof into a patient's body. It is to be understood that a reference to a “non-bendable” porous plug, throughout the specification and the claims, can refer to absolute rigidity that will not allow the porous plug 120 to bend at all, or to a modest degree of bendability, for example up to (no more than) 5°, 10°, or 15°, relative to the central longitudinal axis Cₐ. The bendability of the porous plug may be tested by any method known in art. Since the geometry of a porous plug may vary and influence bendability, a standardized bendability testing procedure proposed herein can optionally rely on bendability test perform on a rectangular cuboid sample of the porous material from which the porous plug is formed. FIG. 11A shows an exemplary rectangular cuboid porous sample 200 that can be used for testing bendability.

The rectangular cuboid sample 200 is made of the material from which the porous plug 120 is formed, and is porous with pore size and density similar to those of the corresponding porous plug 120. Therefore, the rectangular cuboid sample 200 comprises a plurality of pores as the porous plug. Alternatively, it can be said that rectangular cuboid sample 200 has the same porosity as the porous plug. The length×width×height of the rectangular cuboid porous sample can optionally be about 50×10×6 mm. In some examples, a lumen 220 is further comprised in the rectangular cuboid porous sample 200, having a diameter of about 1.5 mm. and extending from a first end 202 of the rectangular cuboid porous sample 200 along a length of about 45 mm, terminating at a lumen distal end 222 that is axially distanced from the second end 204 of the sample 200.

FIG. 11B schematically shows a side view of a rectangular cuboid porous sample 200 subjected to a bending force F_{B} applied to a free end thereof, such as against the upper face 206 of the sample 200, during a bendability test procedure. Bendability of the rectangular cuboid porous sample is assessed by measuring the angle of deflection a of the second end 204 relative to its horizontal pre-bent orientation, upon application of the bending force F_{B} on the second end 204 in a direction perpendicular to the upper face 206, as illustrated in FIG. 11B, while the first end 202 is affixed in position. In some examples, a threshold deflection angle α is defined to assess whether the rectangular cuboid porous sample 200 meets bendability criteria, such that when a bending force F_{B} applied to the second (or free) end 204 of the sample is equal to or greater than 10N, the deflection angle α is not greater than 10 degrees.

In some examples, the deflection angle α is not greater than 10 degrees when a bending force F_{B} of about 12N is applied to the second end of the sample. In some examples, the deflection angle α is not greater than 10 degrees when a bending force F_{B} of about 15N is applied to the second end of the sample. In some examples, the deflection angle α is not greater than 10 degrees when a bending force F_{B} of about 20N is applied to the second end of the sample. In some examples, the deflection angle α is not greater than 10 degrees when a bending force F_{B} of from about 10N to about 100N is applied to the second end of the sample. In some examples, the deflection angle α is not greater than 10 degrees when a bending force F_{B} of from about 10N to about 80N is applied to the second end of the sample. In some examples, the deflection angle α is not greater than 10 degrees when a bending force F_{B} of from about 10N to about 60N or from 10N to about 40N, or from 20N to about 60N or from 20N to about 40N is applied to the second end of the sample.

It is to be understood that a reference to a “non-squeezable” porous plug, throughout the specification and the claims, can refer to absolute stiffness that will not allow the porous plug 120 to be squeezed at all, or to a modest degree of squeezability, for examples by being able to reduce the size of a maximal plug outer dimension by no more than 5% or 10% relative to its free state. The terms “squeezable” and “compressible” are used herein interchangeably. The compressibility of the porous plug may be tested by any method known in art. Since the geometry of a porous plug may vary and influence compressibility, a standardized compressibility testing procedure proposed herein can optionally rely on a bendability test performed on a rectangular cuboid sample of the porous material from which the porous plug is formed, which can be similar to the rectangular cuboid porous sample 200 described above with respect to FIG. 11A.

FIG. 11C shows a cross-sectional view of the rectangular cuboid porous sample 200 along line 11C-11C of FIG. 11A, subjected to a compressive force F_{C} that can be applied thereon from any direction of the sample's cross-section. Compressibility of the rectangular cuboid porous sample is assessed by measuring the change in cross-sectional distance between opposite sides against which the compressive force F_{C} is applied, divided by the original distance between the same sides. In some examples, a compressibility threshold, measured in % change in the cross-sectional distance between opposite sides or faces of the tested sample, is defined to assess whether the rectangular cuboid porous sample meets compressibility criteria, such that when a compressive force F_{C} applied on the sample's cross section is equal to or greater than 10N, the sample is not compressed by more than 10%. In some examples, the sample is not compressed by more than 10% when a cross-section thereof is subjected to a compressive force F_{C} of about 12N. In some examples, the sample is not compressed by more than 10% when a cross-section thereof is subjected to a Fe compressive force of about 15N. In some examples, the sample is not compressed by more than 10% when a cross-section thereof is subjected to a compressive force F_{C} of about 20N. In some examples, the sample is not compressed by more than 10% when a cross-section thereof subjected to a compressive force F_{C} of about 20N.

In some examples, the sample is not compressed by more than 10% when a cross-section thereof subjected to a compressive force F_{C} of from about 10N to about 100 N. In some examples, the sample is not compressed by more than 10% when a cross-section thereof is subjected to a compressive force F_{C} of from about 10N to about 80N. In some examples, the sample is not compressed by more than 10% when a cross-section thereof is subjected to a compressive force F_{C} of from about 10N to about 60N or from10N to about 40N, or from 20N to about 60N or from 20N to about 40N.

In order to meet compressibility criteria, a first compressive force Fel can be applied in a direction configured to compress the upper face 206 and lower face 208 towards each other, measuring the % change in distance between the upper 206 and lower 208 faces. A second force F_{C}2 can be applied in a direction configured to compress the side faces 210 and 212 towards each other, measuring the % change in distance between the side faces 210, 212. Optionally, the sample can be positioned in a testing apparatus configured to apply compressive force F_{C}1, and then rotated by 90 degrees such that compressive force F_{C}2 can be similarly applied. The magnitude of Fel and F_{C}2 can be optionally identical. While a cross-section at a position of the sample 200 that includes the lumen 220 is illustrated in FIG. 11C, compressibility should be tested for both sections of the sample 200 with and without the lumen, such that compressive forces can be applied (in both directions) on the sections that includes the lumen 220 as shown in FIG. 11C, and similar compressive forces can be then applied on a cross-section between the lumen distal end 222 and the second end 202 of the sample 200 (also in both directions).

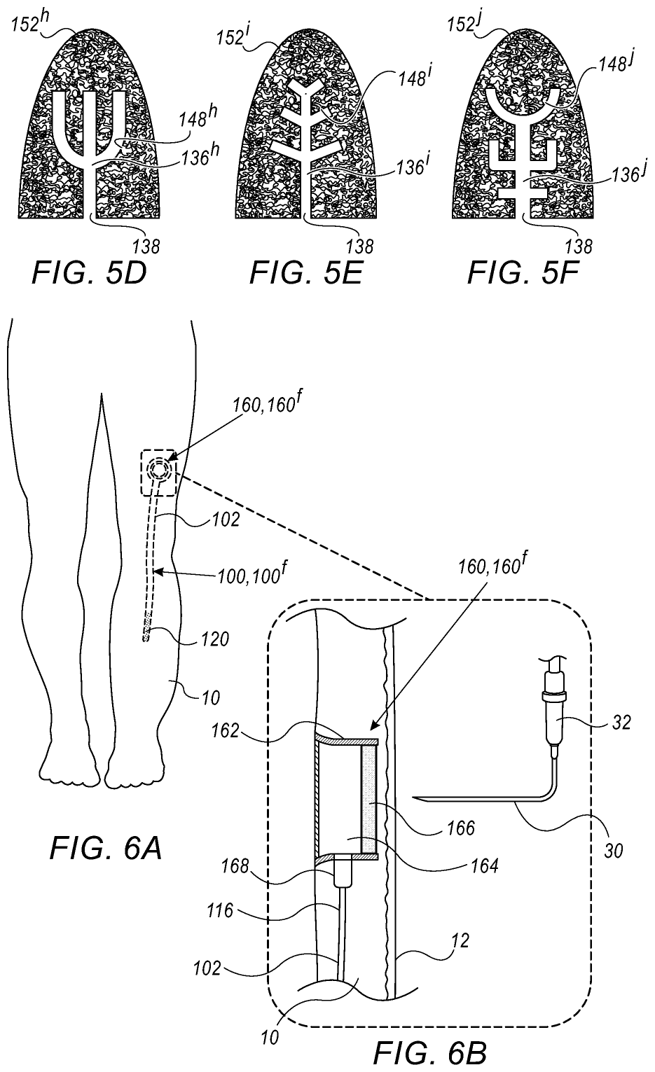

The term “plug outer dimension” refers to a distance between two opposite points along the plug exposed outer surface 128. For cup-shaped or otherwise substantially cylindrical porous plugs 120, such as the exemplary implementations shown in FIGS. 2A-4, a plug outer dimension is the plug outer diameter D_{PO}. A maximal plug outer dimension refers to a maximal plug outer diameter D_{PO}, for example in case the porous plug 120 is formed to have a non-uniform diameter. For a flask-shaped porous plug 120, as will be defined in greater detail below with respect to FIGS. 5A-5C, a plug outer dimension can be either the plug height H_{P} or the plug width W_{P}, defined in greater detail below. In such examples, a maximal plug outer dimension can refer to the maximal values of the plug height H_{P} or the plug width W_{P}, which can be defined at the plug proximal end 150 in the example illustrated in FIGS. 5A-5C.

Any reference to any type of a plug outer dimension, including a plug outer diameter D_{PO}, throughout the specification and the claims, refers to an outer dimension (e.g., outer diameter) of the porous plug 120 in a free state, when not subjected to any external compressive forces, such as when surrounded by an outer atmosphere prior to implantation into a patient's body.

In some examples, the plug total length L_{P} may be designed according to structural characteristics and other dimensions of the drainage device 100. Considerations regarding the length L_{P} can include the width of the plug or plug outer diameter D_{PO}, the width of hollow parts of the plug, and eventually the pressure required for suction of bodily fluids through the porous plug. In some examples, the plug total length L_{P} is from about 0.1 to about 10 centimeters (cm). In some examples, the plug total length L_{P} is from about 1 to about 10 cm. In some examples, the length L_{P} is from about 3 to about 7 cm, from about 3 to about 6 cm, from about 4 to about 6 cm, from about 5 to about 6 cm, from about 1 to about 4 cm, from about 2 to about 4 cm, about 3 cm, or about 5 cm.

In some examples, the plug total length L_{P} is greater than the plug outer diameter D_{PO}. In some examples, the plug total length L_{P} is at least two times as great as the plug outer dimension, such as plug outer diameter D_{PO}. In some examples, the plug total length L_{P} is at least three times as great as the plug outer dimension, such as the plug outer diameter D_{PO}. In some examples, the plug total length L_{P} is at least five times as great as the plug outer dimension, such as the plug outer diameter D_{PO}. It is to be understood that when a porous plug 120 has a non-uniform outer dimension, such as a non-uniform outer diameter, numerical values or size relationships that refer, throughout the specification and the claims, to a plug outer dimension such as plug outer diameter D_{PO}, refer to the maximal plug outer dimension, such as a maximal plug outer diameter D_{PO}, which can be, in some examples, at the plug proximal end 150 (as in the exemplary implementations illustrated in FIGS. 3-5C), and in some examples, at the plug medial portion (as in the exemplary implementation illustrated in FIG. 2B).

In some examples, the tube lumen diameter D_{TL} is in the range from about 0.01 to about 3 cm. In some examples, the diameter D_{TL} is from about 0.05 to about 1 cm. In some examples, the diameter D_{TL} is from about 0.07 to about 0.5 cm. In some examples, the diameter D_{TL} is from about 0.07 to about 0.5 cm. In some examples, the diameter D_{TL} is from about 1 to about 3 millimeters (mm). In some examples, the diameter D_{TL} is from about 1.5 to about 2.5 mm. In some examples, the diameter D_{TL} is from about 1.5 to about 2 mm or about 2 mm.

In some examples, the tube outer diameter D_{TO} is adapted to be used within the human body. In some examples, the tube outer diameter D_{TO} is from about 0.01 to about 3 cm. In some examples, the diameter D_{TO} is from about 0.05 to about 1 cm. In some examples, the diameter D_{TO} is from about 0.07 to about 0.5 cm. In some examples, the diameter D_{TO} is from about 0.07 to about 0.5 cm. In some examples, the diameter D_{TL} is from about 1.5 to about 4 mm. In some examples, the diameter D_{TO} is from about 2 to about 4 mm. In some examples, the diameter D_{TO} is from about 2.5 to about 3.5 mm or about 3 mm.

Any exemplary drainage device 100 disclosed herein is intended for use in a mammal or human body. Therefore, any example of a drainage device 100 disclosed herein is an implantable device. The term “implantable” as used herein means that at least part of the device may be or is implanted into a living body, such as, but not limited to, into the human body. Subsequently, all parts and elements of any exemplary drainage device 100 that are configured to contact or be implanted in a mammal or human body (as well as the body of any other organism of interest), including, for example, any porous plug 120 and/or drainage tube 102 attached thereto, are biocompatible, including by being made from biocompatible materials, and are sterile. The terms “sterile drainage device” and “drainage device”, as used throughout the specification and the claims, are interchangeable. The term “sterile” is one known in the art and may generally refer to a property of an arbitrary object of being at least to a large extent free from all forms of life and/or other biological agents such as prions, viruses, fungi, bacteria or spore forms. Thus, the sterile object may be treated by at least one sterilization process that one or more of reduces, eliminates or deactivates the forms of life and/or of the other biological agents. Drainage device 100 can be in conformity with any standards as required by a government regulatory agency, such as United States Food and Drug Administration (FDA) or European Medicines Agency. Examples of such standards are EN 556-1 and EN 556-2.

In some examples, the porous plug 120 is made of a biocompatible ceramic. In some examples, the porous plug 120 is made of a biocompatible glass. In some examples, the porous plug 120 is made of biocompatible polymer. In some examples, the porous plug 120 is made of a biocompatible metal, such as a metallic foam. Combination of such materials from which the porous plug 120 can be formed are similarly contemplated. In some examples, the porous plug 120 is shaped as a porous cup.

Various exemplary implementations for drainage device 100 and/or components thereof can be referred to, throughout the specification, with superscripts, for case of explanation of features that refer to such exemplary implementations. It is to be understood, however, that any reference to structural or functional features of any device or component, without a superscript, refers to these features being commonly shared by all specific exemplary implementations that can be also indicated by superscripts. In contrast, features emphasized with respect to an exemplary implementation of any device or component, including drainage device 100 and/or a porous plug 120 thereof, referred to with a superscript, may be optionally shared by some but not necessarily all other exemplary implementations. For example, drainage device 100ª, which is indicated in FIG. 1, is an exemplary implementation of drainage device 100, and thus includes all of the features described for drainage device 100 throughout the current disclosure, with the exception that while a drainage device 100 can be partially or fully implanted in a patient's body, the drainage tube 102 of drainage device 100^{a} comprises an implantable tube portion 104 and an extracorporeal tube portion 106, wherein the extracorporeal tube portion 106 can be configured for connection with an external device configured to apply suction to drain bodily fluids through the porous plug 120 and drainage tube 102.

As described above, the drainage device is aimed to allow drainage of bodily fluids from a human organ, such as a swollen limb, without clogging. In order to facilitate drainage, the bodily fluids can be, in some examples, suctioned by a pump. In some implementations, the drainage device 100^{a} can be connected to an external pump 20. For example, external pump 20 can be a pump present in a hospital or other medical facility, such that a patient, having the implantable tube portion 104 of drainage device 100^{a} implanted in its body, can periodically connect the extracorporeal tube portion 106 to an external pump 20 to initiate removal of excess bodily fluids. In some examples, the extracorporeal tube portion 106 can include a connector 108 for connecting with a corresponding external pump connection port 22. In some examples, the extracorporeal tube portion 106 can include a removable cover (not shown) configured to seal the proximal end of the drainage tube 102 when not connected to a pump, and can be removed prior to attaching the drainage tube 102 to the pump. The suction power applied by a pump, such as external pump 20, may be adapted according to the site in which the drainage device is implanted, the size of the plug, its porosity etc.

The term “suction”, as used herein, means the flow of fluid towards a partial vacuum or region of lower pressure. The pressure gradient between this region and the ambient pressure will propel matter toward the low-pressure area. The terms “suction means” and “source of suction”, as used herein, encompass any device capable of drawing a fluid through a conduit, lumen, pipe, passage, etc. Such devices may include, but are not limited to, vacuum pumps, suction pumps, syringes, vacuum blowers, suction systems/plenums commonly found in surgical suites, etc.

As used herein, the term “fluid communication” with respect to two or more components, means that such components are interconnected by a lumen, pipe, conduit, passage, etc. that allows and/or facilitates the passage of fluid therethrough from one component to the other component.

It should be noted that when an external suction means, such as an external pump or syringe, is connected, it may be replaced by a flushing means for, or may include reversed functionality that can be used for, flushing the porous plug and/or the organ in which the plug is implanted, by reversing the direction of fluid streaming from the flushing source towards the porous plug. Flushing fluid utilized in such instances can include any suitable liquid (such as saline) or gas.

FIGS. 2A-2B illustrate an exemplary drainage device 100^{b}, which is an exemplary implementation of drainage device 100, and thus includes all of the features described for drainage device 100 throughout the current disclosure, except that the drainage device 100^{b} comprises a cup-shaped porous plug 120^{b} that includes a plug lumen 136 extending at least through the plug proximal portion 126^{b} and the plug medial portion 124^{b}. Plug lumen 136 can distally extend from the plug proximal end 150 along a plug lumen length L_{PL}, which is shorter than the plug total length L_{P} (i.e., L_{P}>L_{PL}). The plug lumen 136 can be cylindrical in shape, defining a plug lumen diameter D_{PL}, though it is to be understood that other shapes, which are not necessarily cylindrical, are also contemplated for either the porous plug 120 (for example, the exposed outer surface 128 around plug medial portion 124 can be non-cylindrical) and/or the plug lumen 136.

The plug lumen 136 is in fluid communication with, and can be continuous with, the tube lumen 110. The plug inner uncovered surface 148 is exposed to, and is in fluid communication with, the tube lumen 110, either directly or via plug lumen 136. In some examples, at least a portion of the plug inner uncovered surface 148 is exposed to, and is in fluid communication with, the plug lumen 136. At least some of the pore inner openings 132 are facing, and are in fluid communication with, the plug lumen 136. Thus, when suction force is applied to a tube proximal portion 116, bodily fluid surrounding the porous plug 120^{b} flow, through the pore outer openings 132, the pore interconnected channels 130, and the pore inner openings 134, into the plug lumen 136, and are further suctioned from the plug lumen 136 into the tube lumen 110.

In some examples, the plug lumen length L_{PL} is from about 0.1 to about 10 cm. In some examples, the plug lumen length L_{PL} is from about 1 to about 10 cm. In some examples, the plug lumen length L_{PL} is from about 2 to about 10 cm. In some examples the length L_{PL} is from about 3 to about 7 cm, from about 3 to about 6 cm, from about 4 to about 6 cm, from about 5 to about 6 cm, or about 5 cm. In some examples, the length L_{P} is from about 5 to about 6 cm and the length L_{PL} is from about 4.5 to about 5.8 cm. In some examples, the porous plug 120^{a} has a uniform thickness along its plug medial portion 124^{b} and optionally along the plug distal portion 122^{b}, defined as the thickness between the plug exposed outer surface 128 and the plug lumen 136 (or plug inner uncovered surface 148) at each position of the plug medial portion 124^{b} and plug distal portion 1225. In some examples, the plug lumen length L_{PL}, is not less than 80% of the plug total length L_{P}. In some examples, the plug lumen length L_{PL} is not less than 70% of the plug total length L_{P}.

In some examples, the plug lumen diameter D_{PL} is equal to or greater than 30% of the plug outer diameter D_{PO}. In some examples, the plug lumen diameter D_{PL} is equal to or greater than 50% of the plug outer diameter D_{PO}. In some examples, the plug lumen diameter D_{PL} is equal to or greater than 30% of the tube lumen diameter D_{TO}. In some examples, the plug lumen diameter D_{PL} is equal to or greater than 50% of the tube lumen diameter D_{TO}.

In some examples, the plug lumen length L_{PL} is greater than the plug lumen diameter D_{PL}. In some examples, the plug lumen length L_{PL}, is at least two times as great as the plug lumen diameter D_{PL}. In some examples, the plug lumen length L_{PL} is at least three times as great as the plug lumen diameter D_{PL}. In some examples, the plug lumen length L_{PL} is at least five times as great as the plug lumen diameter D_{PL}.

The cup-shaped porous plug 120^{b} is further shown to have a rounded atraumatic plug distal portion 122, which can optionally be hemispherical as in the illustrated example, or have other curved shapes that may be tapering from the plug medial portion 124 towards a curved plug distal end 152.

The tube distal portion 112 can be attached to the porous plug 120 in a variety of manners, including by being attached thereto directly or via one or more intermediate components. In some examples, the tube distal portion 112 can overlap to a certain length with the plug proximal portion 126. In some examples, the tube distal portion 112 can be attached to an outer surface of the porous plug 120. In some examples, the tube distal portion 112 can be disposed around the plug proximal portion 126. In some examples, the tube distal portion 112 can be attached to an inner surface of the porous plug 120. In some examples, the tube distal portion 112 can be disposed inside the plug proximal portion 126, such as by extending through a proximal opening of the porous plug 120. FIG. 2B illustrates an exemplary attachment configuration by which the tube distal portion 112 is disposed around the plug proximal portion 126^{b}. In some examples, the porous plug 120 can define a plug outer step 140 around the plug proximal portion 126, such as plug outer step 140 around the proximal portion 126^{b} of exemplary plug 120^{b}, on which the tube distal portion 112 can be fitted and attached to.

In some examples, the radial depth of the plug outer step 140 is substantially equal to the thickness of the drainage tube 102, such that when the tube distal portion 112 is disposed inside the plug outer step 140, the outer surface of the drainage tube 102 is flush with the plug exposed outer surface 128 as illustrated in FIG. 2B. This has the advantage of avoiding any distally oriented steps protrusions that may otherwise interfere with insertion of the drainage device 100^{b} into a patient body during implantation. Attachment of a tube distal portion 112 to a porous plug 120, according to any of the exemplary configurations disclosed herein, can be achieved by any methods known in the art, such as by gluing, bonding, welding, press-fitting, friction-fitting, threading, and the like.

In some implementations, a portion of the porous plug 120 can be covered by the tube distal portion 112, which in turn blocks the pore outer openings 132 at the covered region. Thus, a plug exposed outer surface 128 refers to an outer surface of the porous plug 120 which remains uncovered, such that the pore outer openings 132 defined thereover remain exposed and unsealed. Porous plug 120^{b} is shown in FIG. 2B to have its plug proximal portion 126^{b} covered by the tube distal portion 112, thereby sealing any pore outer openings 132 of the plug proximal portion 126^{b} that may have been exposed prior to attachment of the drainage tube 102 to the porous plug 120^{b}. In this case, the plug exposed outer surface 128^{b} of porous plug 120^{b} is defined only along the plug distal portion 122^{b} and plug medial portion 124^{b}.

In some implementations, the tube lumen diameter D_{TL} is greater than the plug lumen diameter D_{PL}, as shown for example for drainage device 100^{b} in FIG. 2B. This in turn forms a proximally facing surface of the porous plug 120^{b} at the plug proximal end 150. The term “proximally oriented surface”, as used herein, refers to a surface that is orthogonal to the central longitudinal axis Cₐ, and in the case of drainage device 100^{b} illustrated in FIG. 2B, forms a step-like configuration facing the tube lumen 110. In the illustrated example, this proximally-oriented surface of porous plug 120^{b} can also include pore inner opening 134, thereby forming part of the plug inner uncovered surface 148^{b} along with the inner surface surrounding the plug lumen 136.

FIG. 3 shows an exemplary drainage tube 100^{c} comprising a porous plug 120^{c}.

Drainage device 100^{c} is an exemplary implementation of drainage device 100, and thus includes all of the features described for drainage device 100 throughout the current disclosure, except that the drainage device 100^{c} comprises a cup-shaped porous plug 120^{c} that does not include a plug lumen 136 extending through any of its plug medial portion 124^{c} and its plug distal portion 122^{c}. Porous plug 120^{c} can be also referred to as a full-body plug, devoid of any cavities or lumens formed inside or along its plug medial portion 124^{c} and its plug distal portion 122^{c}, other than the pores (i.e., other than the pore outer openings 132, pore inner opening 134, and pore interconnected channels 130). In some examples, as shown in FIG. 3, a porous plug 120^{c} can still include a plug lumen 136, that extends from the plug proximal end 150 along the plug proximal portion 126^{c}, but terminates at, or proximal to, the plug medial portion 124^{c}.

In some examples, the porous plug 120 can define a plug proximal opening 138 at the plug proximal end 150. A lumen 136 of the porous plug 120 can distally extend from the plug proximal opening 138. In some examples, the plug proximal opening 138 has a plug opening diameter D_{PH} (indicated, for example, in FIG. 5A), which can be equal to or slightly greater than the plug lumen diameter D_{PL}, the tube distal portion 112 can be inserted, through the plug proximal opening 138, into the plug lumen 136. In some examples, the plug outer diameter D_{PO} is greater than the tube outer diameter D_{TO}.

Porous plug 120^{c} is shown to have a uniform plug outer diameter D_{PO} along the plug medial portion 124^{c} and the plug proximal portion 126^{c}, which is greater than the tube outer diameter D_{TO}. This in turn forms a proximally facing surface of the porous plug 120^{c} at the plug proximal end 150, which, unlike the proximally oriented surface illustrated in FIG. 2B for porous plug 120^{b}, is not facing the tube lumen but is rather disposed around the drainage tube 102, exposed to the environment surrounding the tube distal portion 112. In the illustrated example, this proximally-oriented surface of porous plug 120^{c} can also include pore outer openings 132, thereby forming part of the plug exposed outer surface 128^{c} along with the outer surface surrounding the plug proximal portion 126″, the plug medial portion 124^{c} and the plug distal portion 122^{c}.

In some implementations, the tube distal portion 112 can extend along the entire length of the plug lumen 136, as shown for drainage device 100^{c} in FIG. 3. This in turn can cover the inner surface circumferentially surrounding plug lumen 136, thereby sealing any pore inner openings 134 of the plug proximal portion 126^{c} that may have been uncovered prior to attachment of the drainage tube 102 to the porous plug 120^{c}. In this case, the plug inner uncovered surface 148″ of porous plug 120^{c} is defined only by proximally oriented surface of the porous plug 120^{c} at the level of the tube distal opening 114 (which is also the proximal border of the plug medial portion 124^{c} in the illustrated example), which includes unsealed pore inner openings 134 facing the tube lumen 110.

Advantages associated with full-body plugs, such as porous plug 120^{c}, include simpler manufacturing procedures of the porous plugs and improved structural integrity, which can be of importance in case the porous plug 120 is inserted into a muscular tissue, such as that of a swollen limb (for example, a leg or an arm of the patient). However, the flow paths along the pore interconnected channels 130, from the pore outer openings 132 to the pore inner openings 134, can be significantly longer, for example in comparison to hollow porous plugs of the type shown in FIG. 2B, which can in turn increase resistance to flow, requiring a pump or any other suction device to apply greater suctioning force to drain the fluids through the porous plug.

While FIG. 3 illustrates a porous plug 120 which is a full-body plug, and a drainage tube 102 connected to the porous plug 120 by being inserted thereinto, it is to be understood that these are independent features that can be separated from each other. For example, a drainage tube 102 can be connected to a full-body porous plug 120 in any other manner, including by having its tube distal portion 112 disposed around plug proximal portion 126 in a manner similar to that described for drainage device 100^{b} with respect to FIG. 2B.

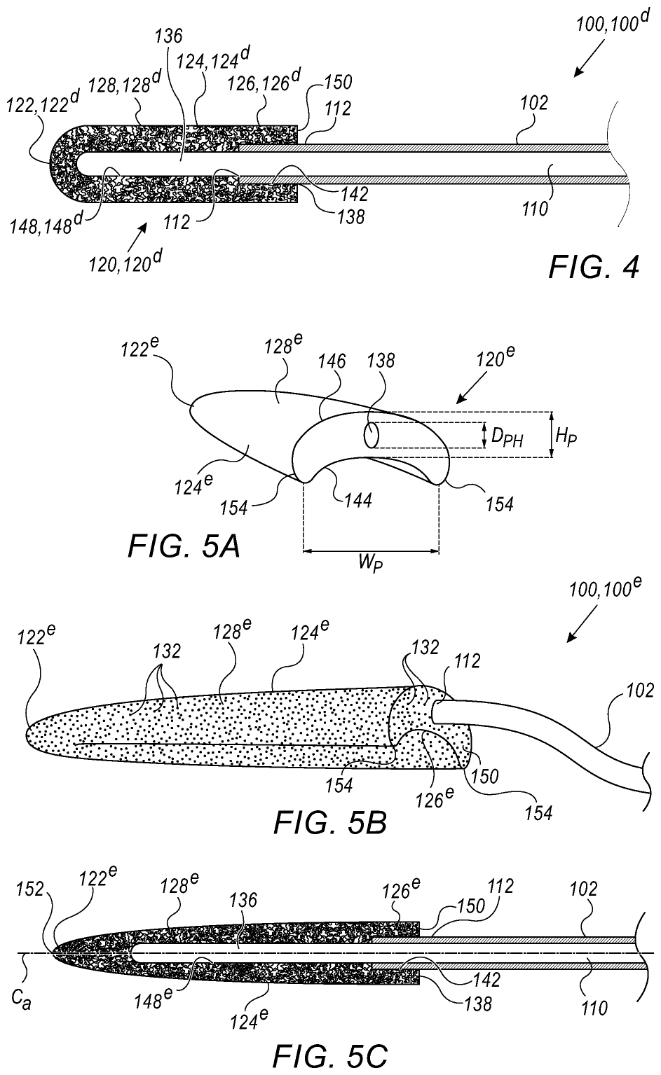

FIG. 4 shows an exemplary drainage device 100^{d} comprising a porous plug 120^{d}.

Drainage device 100^{d} is an exemplary implementation of drainage device 100, and thus includes all of the features described for drainage device 100 throughout the current disclosure, except that the drainage device 100^{d} comprises a drainage tube 102 which is inserted into a hollow cup-shaped porous plug 1200 that includes a plug lumen 136 extending through both of its plug proximal portion 126^{d} and plug medial portion 124^{d}.

The exposed outer surface 1284 of porous plug 120^{d} can be generally similar to that described above with respect to porous plug 120^{c}. Porous plug 120^{d} is shown to have a uniform plug outer diameter D_{PO} along the plug medial portion 124^{d} and the plug proximal portion 1264, which is greater than the tube outer diameter D_{TO}, thus forming a proximally facing surface of the porous plug 120^{d} at the plug proximal end 150 exposed to the environment surrounding the tube distal portion 112. In the illustrated example, this proximally-oriented surface of porous plug 120^{d} can also include pore outer opening 132, thereby forming part of the plug exposed outer surface 128^{d} along with the outer surface surrounding the plug proximal portion 126^{d}, plug medial portion 124^{d} and plug distal portion 122^{d}.

In some examples, the porous plug 120 can define a plug inner step 142 inside the plug proximal portion 126, such as plug inner step 142 inside the proximal portion 126^{a} of exemplary plug 120^{d}, configured to accommodate the tube distal portion 112 therein, such that the tube distal portion 112 can be inserted into plug inner step 142 and attached thereto. In some examples, the radial depth of the plug inner step 142 is substantially equal to the thickness of the drainage tube 102, such that when the tube distal portion 112 is disposed inside the plug inner step 142, the inner surface of the drainage tube 102 (around tube lumen 110) is flush with the plug inner uncovered surface 148 (around plug lumen 136), as illustrated in FIG. 4. In the illustrated example, the plug opening diameter D_{PO} can be equal to the plug lumen diameter D_{PL} plus twice the thickness of the tube distal portion 112.

In some implementations, the tube distal portion 112 can extend along a certain length of the plug lumen 136, such as along a length of the plug lumen 136 extending through the plug proximal portion 126^{d} as shown for drainage device 100^{d} in FIG. 4. This in turn can cover the inner surface circumferentially surrounding plug lumen 136 along the plug proximal portion 1264, thereby sealing any pore inner openings 134 of the plug proximal portion 126^{d} that may have been uncovered prior to attachment of the drainage tube 102 to the porous plug 120^{d}. In this case, the plug inner uncovered surface 148^{d} of porous plug 120^{d} is defined only by the inner surface of the porous plug 120^{d} that surrounds the plug lumen 136 along the plug medial portion 1244, and optionally along the plug distal portion 122^{d}, thus including unsealed pore inner openings 134 facing the plug lumen 136.

FIG. 5A shows a view in perspective of an exemplary porous plug 120^{e}. FIGS. 5B and 5C show a side view in perspective and a cross-sectional view, respectively, of a drainage device 100^{e} comprising the porous plug 120^{e} of FIG. 5A. Porous plug 120^{e} is an exemplary implementation of porous plug 120, and thus includes all of the features described for porous plug 120 throughout the current disclosure, except that the porous plug 120^{e} can be generally hip-flask shaped, having a kidney shaped cross-section at least along the plug medial portion 124^{e}, and optionally also along the plug proximal portion 126^{e}, best visualized for example at the plug proximal end 150 in FIG. 5A-5B.

The portion of the plug exposed outer surface 128^{d} that surrounds the central longitudinal axis Cₐ defines a first surface 144 and a second surface 146, each of which is curved relative to the central longitudinal axis Cₐ. In some examples, the first surface 144 has a concave profile relative to the central longitudinal axis Cₐ, and the second surface 146, which is opposite to the first surface 144, is convex relative to the central longitudinal axis Ca.

In some examples, the radius of curvature of the first surface 144 is different from that of the second surface 146. In the illustrated example, the first surface 144 is shown to have a radius of curvature which is generally greater than that of the second surface 146, which results in both surfaces 144, 146 converging towards each other at their lateral sides 154. As shown, the lateral sides 154 of the porous plug 120^{e}, at which both surfaces 144, 146 converge, can be rounded to avoid any sharp edges that may damage the tissue surrounding the porous plug 120^{e} when implanted. While shown to have different radii of curvature in the illustrated example, it is to be understood that in some examples, the radii of curvature of both the first and second surfaces can be similar, extending generally in parallel to each other, with the porous plug optionally further defining curved (e.g., hemispherical) lateral sides connecting both the first and second surfaces (examples not shown).

The porous plug 120^{e} has a plug height H_{P}, defined as the distance between the first surface 144 and the second surface 146, at the lateral position of the central longitudinal axis Cₐ. The term “lateral position”, as used herein, refers to a position that can vary between the lateral sides 154. A lateral position of the central longitudinal axis Cₐ is a position at a midpoint between both lateral sides 154, which is also the position along which the cross-sectional view of FIG. 5C is taken. When the radii of curvature of both surfaces 144, 146 are different, as in the illustrated example, the distance between both surfaces is maximal at the lateral position of the central longitudinal axis Cₐ, by which the plug height H_{P} is defined, and is reduced in size towards the lateral sides 154. In some examples, a maximal plug height H_{P} is from about 2 to about 15 mm, from about 3 to about 10 mm, from about 3 to about 7 mm, or about 5 mm.

The porous plug 120^{e} has a plug width W_{P}, defined as the distance between both lateral sides 154. Plug width W_{P} is perpendicular to plug height H_{P}. In some examples, the maximal plug width W_{P} is from about 5 to about 30 mm. In some examples, the maximal width W_{P} is from about 5 to about 20 mm, from about 5 to about 15 mm, from about 7 to about 12 mm, or about 10 mm.

In some examples, a porous plug 120 can taper in the distal direction, at least along its plug medial portion 124. In some examples, the porous plug 120 can taper from the plug proximal end 150 to the plug distal end. In the case of cylindrical or cup-shaped porous plugs, the plug outer diameter D_{PO} can vary from a relatively greater diameter at the plug proximal end to a narrower diameter towards the plug distal end (example not shown). In the case of a flask-shaped porous plug 120^{e}, the plug height H_{P} can decrease towards the plug distal end. The plug width W_{P} can similarly decrease in size in the distal direction. While a tapering plug 120^{e} is illustrated, it is to be understood that in some examples, a flask-shaped porous plug does not necessarily have to taper, and may have a uniform plug height H_{P} along any of its plug medial portion and/or its plug proximal portion (example not illustrated).

In the illustrated example, the porous plug 120^{e} is shown to have a plug proximal opening 138 leading to a plug lumen 136 extending through the plug proximal portion 126^{c} and the plug medial portion 124^{c}. As shown in the illustrated example, the plug lumen 136 can have a uniform plug lumen diameter D_{PL}, at least along the plug medial portion 124^{c}, even if the porous plug 120^{e} is shaped to taper at its outer surface 128^{c}. In some examples, the plug lumen 136 can be differently sized and shaped, including following a shape that can follow the shape of the outer surface 128^{c}, and/or sized to as to narrow in the distal direction.

In some examples, the plug total length L_{P} of porous plug 120^{e} is from 10 to 100 mm, from about 20 to about 80 mm, from about 30 to about 70 mm, from about 40 to about 60 mm, from about 45 to about 55 mm, or about 50 mm.

In some examples, a maximal plug width W_{P} is from about 5 to about 15 mm, or about 10 mm, a maximal plug height H_{P} is from about 3 to about 10 mm, or about 5 mm, and the plug total length L_{P} is from about 40 to about 60 mm, or about 50 mm.

In some examples, the maximal plug width W_{P} is greater than the maximal plug height H_{P}. The plug width W_{P} and plug height H_{P} can have maximal values at the plug proximal end 150, as illustrated in FIGS. 5A-5B. In some examples, the maximal plug width W_{P} is at least two times as great as the maximal plug height H_{P}. In some examples, the maximal plug width W_{P} is at least three times as great as the maximal plug height H_{P}.

In some examples, at least one of the maximal plug height H_{P} and/or maximal plug width W_{P} is greater in size than the tube outer diameter D_{TO}. Porous plug 120^{e} is shown to have both a maximal plug height H_{P} and a maximal plug width W_{P} which are greater in size, at the plug proximal end 150, than the tube outer diameter D_{TO}, thus forming a proximally facing surface of the porous plug 120^{e} at the plug proximal end 150 exposed to the environment surrounding the tube distal portion 112. In the illustrated example, this proximally-oriented surface of porous plug 120^{e} can also include pore outer opening 132, thereby forming part of the plug exposed outer surface 128^{e} along with the outer surface surrounding the plug proximal portion 126^{e} plug medial portion 124^{e} and plug distal portion 122^{e}.

While porous plug 120^{e} is illustrated as a hollow porous plug that includes a plug lumen 136 extending through the plug medial portion 124^{e}, it is to be understood that this is shown by way of illustration and not limitation, and that a non-cylindrical porous plug, such as a flask-shaped porous plug, can be implemented as a full-body plug, devoid of any cavities or lumens formed inside or along its plug medial portion and its plug distal portion, other than the pores.

It is to be understood that any porous plug disclosed herein, including cup-shaped porous plug 120^{b} and 120^{d}, or curved porous plug 120^{e}, can include an internal plug lumen 136 having any shape, including the generally cylindrical shapes of the plug lumens illustrated in FIGS. 2B, 4 and 5C, as well as non-linearly cylindrical shapes. Examples of optional non-linear cylindrical shapes of plug lumen 136 are illustrated in FIGS. 5D, 5E and 5F, showing exemplary hollow porous plugs 100^{b}, 100ⁱ and 100ⁱ having branched plug lumens 136^{b}, 136ⁱ and 136^{j}, respectively. Branched plug lumens can advantageously increase the total area of the plug inner uncovered surface, such as surfaces 148^{b}, 148ⁱ and 148^{j} in the illustrated examples.

While drainage device 100^{e} is illustrated to include a drainage tube 102 inserted into a porous plug 120^{e}, and the porous plug 120^{e} is illustrated to include a plug inner step 142 that accommodates the tube distal portion 112, it is to be understood that this is shown by way of illustration and not limitation, and that drainage tube 102 can be attached to a non-cylindrical porous plug, such as a flask-shaped porous plug, in any other suitable manner, including disposed at least partially around at least a portion of the plug proximal portion (examples not shown). According to some examples, the connection between the drainage tube 102 and the porous plugs 120 may be performed using a suitable biocompatible glue.

While some exemplary porous plugs, such as porous plugs 120^{c}, 120^{d}, or 120^{e}, are illustrated to include a stepped shape at their plug proximal ends 150, it is to be understood that this is shown by way of illustration and not limitation, and that in some examples, the outer edges of a plug proximal end 150 can be rounded or chamfered.

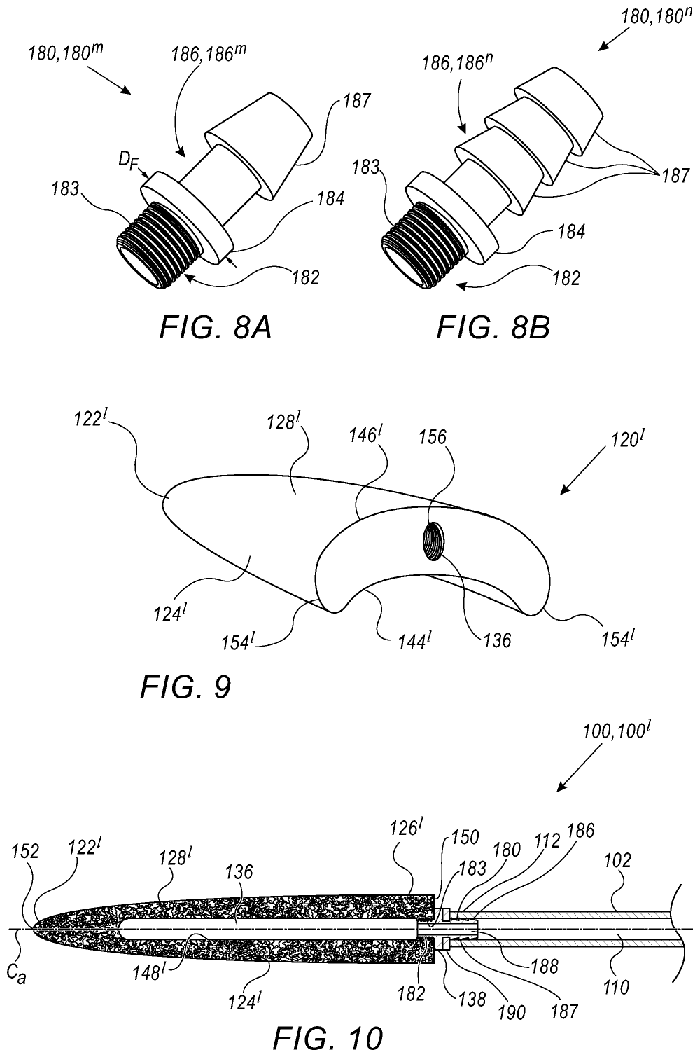

While some exemplary drainage devices disclosed herein are illustrated to have the drainage tube 102 directly attached to the porous plug 120, optionally by insertion of the drainage tube 102 into and through the plug proximal opening 138, it is to be understood that any other suitable manner of attachment is contemplated. In some examples, a hollow connector can be used to ensure proper leak-proof connection of the drainage tube 102 to the porous plug 120. In some examples, a threaded barbed connector 180, examples of which are shown in FIGS. 8A and 8B, can be used to secure the drainage tube 102 to the porous plug 120. As shown in FIGS. 8A-8B, a threaded barbed connector 180 includes a distal extension 182 having an outer threading 183, and a proximal extension 186 having one or more barbs 187. A flange 184 can be optionally disposed between the proximal extension 186 and the distal extension 182. FIG. 8A shows an exemplary threaded barbed connector 180^{m} having a proximal extension 186^{m} equipped with a single tapered barb 187, and FIG. 8B shows an exemplary threaded barbed connector 180ⁿ having a proximal extension 186ⁿ equipped with three tapered barbs 187. It is to be understood that any other number of barbs 187 is contemplated.

FIG. 9 shows an exemplary porous plug 120^{l} of an exemplary drainage device 100^{l} illustrated in FIG. 10. Porous plug 120^{l} is an exemplary implementation of porous plug 120, and thus includes all of the features described for porous plug 120 throughout the current disclosure, except that the porous plug 120^{l} further defined an inner threading 156 along a proximal portion of the plug lumen 136, configured to mate with the outer threading 183 of the threaded barbed connector 180, as shown in FIG. 10. It is to be understood that a hip-flask shaped porous plug 120^{l}, similar to exemplary porous plug 120^{e} described above, is shown by way of illustration and not limitation, and that any other type of porous plug 120 disclosed herein can include the inner threading 156.

As further shown in FIG. 10, the barbed proximal extension 186 is configured to grip onto the tube distal portion 112, while the flange 184 can abut the plug proximal end 150. In some examples, a sealing member 190, such as an O-ring, is further added between the distal lip of the drainage tube 102 and the flange, as illustrated in FIG. 10. A similar sealing member 190 (e.g., O-ring) can be positioned, in some examples, between the flange 184 and the plug proximal end 150. A lumen 188 defined by the hollow threaded barbed connector 180 is in fluid communication with the tube lumen 110 and the plug lumen 136. While a circular flange 184 is illustrated, it is to be understood that any other shape is contemplated. For example, a flange 184 can be provided in the form of a nut to enable securement of the threaded barbed connector 180 by a suitable rotation tool (not shown). In some examples, the flange 184 can have an atraumatic shape. The flange 184 can define an outer diameter DF that is equal to or less than the plug outer diameter D_{PO} or of the plug height H_{P}, so as to avoid formation of a step-like radial extension that can interfere with distal advancement of the porous plug 120 through the tissue.

While barbs 187 are illustrated in the examples shown in FIGS. 8A-8B, it is to be understood that a proximal extension 186 can include any other type of fastening feature(s). For example, a proximal extension 186 can include an outer threading, configured to mate with an inner threading formed in a tube distal portion 112 (not shown). Moreover, it is to be understood that a threaded barbed connector 180 is shown by way of example only, and that any other suitable type of connector can be used to form a sealed secured coupling between the porous plug 120 and the drainage tube 102.

While some exemplary porous plugs, such as porous plugs 120^{c} or 120^{d}, are shown to have a uniform plug outer diameter D_{PO} along their plug proximal portions 126, and an exemplary porous plug 120^{e} is shown to have a distally tapering plug proximal portion 126^{c}, it is to be understood that this is shown by way of illustration and not limitation, and that in some examples, the plug proximal portion 126 can taper proximally from the plug medial portion 124 to a narrower plug outer diameter D_{PO} or a smaller plug height H_{P} at the plug proximal end 150 (examples not shown). A proximally tapering plug proximal portion 126 that narrows in size towards the plug proximal end 150 can facilitate easier proximally oriented movement of the porous plug 120 when already inside the patient's body, such as during retrieval of the drainage device 100 from the patient's body.

While some exemplary drainage devices, such as drainage device 100^{c}, 100^{d}, or 100^{e}, show a drainage tube 102 insertable into a porous plug 120 through a plug proximal opening 138 that has a size similar to that of the tube outer diameter D_{TO}, with the porous plug 120 optionally having a plug inner step 142 for receiving the tube distal portion 112, it is to be understood that these are shown by way of illustration and not limitation, and that in some examples, the drainage tube 102 can be inserted into a plug proximal opening 138 that has a plug opening diameter D_{PH} which is less than the tube outer diameter D_{TO}, wherein the porous plug can be provided with or without an inner step. The drainage tube 102 can be made of silicon or other suitable material that can be flexible and somewhat squeezable, allowing the tube distal portion 112 to be squeezed through the narrower plug proximal opening 138, and press-fit in position.

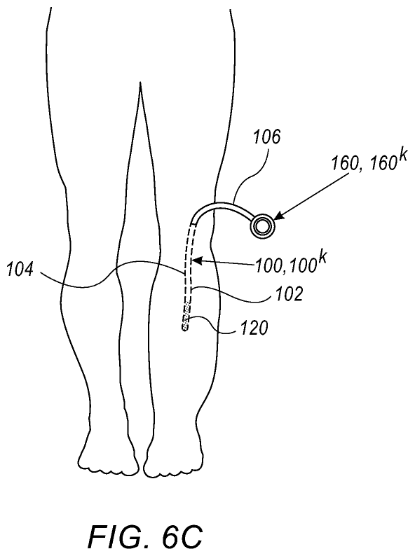

FIGS. 6A-6B show an exemplary drainage device 100^{f}. Drainage device 100^{f} is an exemplary implementation of drainage device 100, and thus includes all of the features described for drainage device 100 throughout the current disclosure, except that the drainage device 100 further comprises an access port 160 attached to the drainage tube 102, such as to the tube proximal portion 116. Access port 160^{f} of drainage device 100^{f} can be an implantable drainage port, such that drainage device 100 is configured to be completely implanted in a patient's body, without including any extracorporeal portions that extend out of the patient's body. FIG. 6A illustrated a drainage device 100^{f} completely implanted in a patient, such as leg 10, though other organs, tissues or cavities in which the drainage device 100^{f} can be implanted are contemplated. FIG. 6B shows an enlarged sectional view of a proximal portion of the drainage device 100^{l}, including the access port 160 and tube proximal portion 116, residing inside the patient's limp.

In some implementations, an implantable access port 160^{f} is located under the skin 12 of the patient, as shown in FIGS. 6A-6B. Access port 160 generally comprise a housing 162 defining a chamber 164 therein, and a self-scaling septum 166 attached to a rim of the housing 162 and enclosing the chamber 164. The chamber 164 is in fluid communication with the tube lumen 110. In some examples, the tube proximal portion 116 can be connected to the housing 162 of implantable access port 160 via a connector, such as a tube coupler 168.

In use, when the drainage device 100^{f} is implanted along with an implantable type of an access port 160^{f}, a needle 30, which can be attached to a drainage bag via a needle hub 32, can be used to puncture through the patient's skin 12 and the self-scaling septum 166 that faces the skin 12, thus gaining access to the chamber 164. In this manner, needle 30 can be periodically used to apply suction to the chamber 164, which in turn causes bodily fluids surrounding porous plug 120 to flow, through the pores of the porous plug 120, into tube lumen 110, and into the chamber 164 and needle 30 therefrom. The self-sealing septum 166 can be a rubber or silicone membrane that seal itself when the needle is removed, to prevent infection or contamination of the site.

FIG. 6C show an exemplary drainage device 100^{k}. Drainage device 100^{k} can be generally similar to any example described above for drainage device 100′, including an access port 160 coupled to the drainage tube 102 and generally structured in a similar manner to that described above with respect to FIGS. 6A-6B, except that the access port 160^{k} of drainage device 100^{k} is configured to remain external to the patient's body. Specifically, the drainage tube 102 can include an implantable tube portion 104 and an extracorporeal tube portion 106, in a similar manner to that described above with respect to drainage device 100^{a}, wherein the external access port 160^{k} is attached to a proximal end of the extracorporeal tube portion 106.

A needle 30 can be used to penetrate through the seal-scaling septum 166 and gain access to the chamber 164 and tube lumen 110 of drainage device 100^{k} in the same manner described above, except that the needle 30 does not need in such instances to penetrate through the patient's skin, as the external access port 160^{k} remains exposed whenever suction therethrough is required.

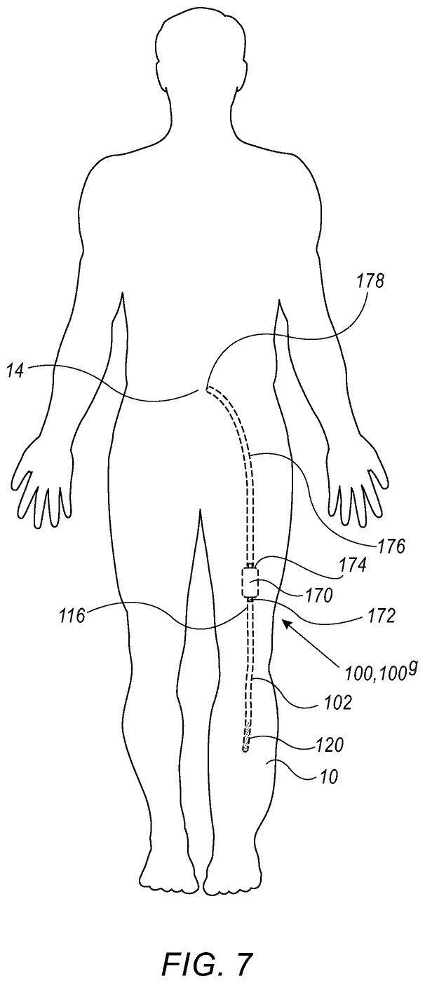

FIG. 7 shows an exemplary drainage device 100^{g}. Drainage device 100^{g} is an exemplary implementation of drainage device 100, and thus includes all of the features described for drainage device 100 throughout the current disclosure, except that the drainage device 100^{g} further comprises an implantable pump 170 attached to the drainage tube 102 and to an implantable outlet tube 176 that terminates at an outlet opening 178. Drainage device 100^{g} is configured to be completely implanted in a patient's body, without including any extracorporeal portions that extend out of the patient's body.

In some examples, the implantable pump 170 can include a pump inlet port 172 to which the drainage tube 102 can be attached. For example, the tube proximal portion 116 can be coupled to the pump inlet port 172. In some examples, the implantable pump 170 can include a pump outlet port 174 to which the implantable outlet tube 176 can be attached. The implantable pump 170 is configured to apply a suctioning force (i.e., negative pressure) to the drainage tube 102, so as to causes bodily fluids surrounding porous plug 120 to flow, through the pores of the porous plug 120, into tube lumen 110, towards the implantable pump 170, and transported from the pump 170, through a lumen of the implantable outlet tube 176, towards and out of the outlet opening. The outlet opening 178 of the implantable outlet tube 176 can be placed in a target location in the patient's body in which the drained fluids can be expelled, such as the abdomen 14 in the illustrated example, or any other organ, tissue or cavity. In some examples, the implantable outlet tube 176 can be connected to a blood vessel and the bodily fluids surrounding the porous plug 120 can be drained to the circulatory system, digestive system, urinary tract, lymphatic system, or any other adequate target system.

It is to be understood that the porous plug 120 of any of the exemplary drainage device 100^{a} described with respect to FIG. 1, drainage device 100^{f} described with respect to FIGS. 6A-6B, or drainage device 100^{g} described with respect to FIG. 7, can be implemented according to any example disclosed herein for porous plugs 120, including cup-shaped plugs or hip-flask shaped plugs, either of which can be a hollow plug or a full-body plug. It is to be understood that attachment of a drainage tube 102 to a porous plug 120 of any of the exemplary drainage device 100^{a} described with respect to FIG. 1, drainage device 100^{f} described with respect to FIGS. 6A-6B, or drainage device 100^{g} described with respect to FIG. 7, can be implemented according to any manner disclosed herein, including having the tube distal portion 112 disposed around an outer surface of the plug proximal portion 126 or inserted through a plug proximal opening 138.

### Some Examples of the Disclosed Implementations

Some examples of above-described implementations are enumerated below. It should be noted that one feature of an example in isolation or more than one feature of the example taken in combination and, optionally, in combination with one or more features of one or more examples below are examples also falling within the disclosure of this application.

Example 1. A sterile drainage device for draining bodily fluids, comprising:
a drainage tube comprising a tube distal portion, the drainage tube defining a tube lumen extending along a central longitudinal axis; and
a porous plug comprising:
   a plug proximal portion attached to the tube distal portion;
   a plug exposed outer surface facing away from the central longitudinal axis;
   a plug inner uncovered surface which is exposed to, and is in fluid communication with, the tube lumen;
   a plurality of pores comprising:
      a plurality of pore outer openings at the plug exposed outer surface;
      a plurality of pore inner openings at the plug inner uncovered surface; and
      a plurality of pore interconnected channels extending through the porous plug, between the pore outer openings and the pore inner openings, such that the pore outer openings are in fluid communication, via the pore interconnected channels and the pore inner openings, with the tube lumen;
      wherein a porous rectangular cuboid sample of the porous plug is not bendable to more than 10° when a bending force of 10N or more is applied to a second end thereof while a first end thereof is affixed in position, wherein the sample is made from the material of the porous plug, comprises a plurality of pores as in the porous plug, has a length×width×height of 50×10×6 mm, and comprises a lumen extending from the first end along a length of 45 mm, the lumen having a diameter of 1.5 mm;
wherein the porous rectangular cuboid sample is not compressible by more than 10% when a compressive force of 10N or more is applied to a cross-section thereof; and
wherein the porous plug is close-ended at a plug distal portion thereof, such that fluid communication between the tube lumen and an environment surrounding the porous plug is achieved solely through the plurality of pores.

Example 2. The sterile drainage device of any example herein, particularly example 1, wherein the bending force applied to the second end of the sample is equal to at least 12N.

Example 3. The sterile drainage device of any example herein, particularly example 1, wherein the bending force applied to the second end of the sample is equal to at least 15N.

Example 4. The sterile drainage device of any example herein, particularly example 1, wherein the bending force applied to the second end of the sample is equal to at least 20N.

Example 5. The sterile drainage device of any example herein, particularly to any one of examples 1 to 4, wherein the compressive force applied to the cross-section of the sample is equal to at least 12N.

Example 6. The sterile drainage device of any example herein, particularly to any one of example 1 to 4, wherein the compressive force applied to the cross-section of the sample is equal to at least 15N.

Example 7. The sterile drainage device of any example herein, particularly to any one of example 1 to 4, wherein the compressive force applied to the cross-section of the sample is equal to at least 20N.

Example 8. The sterile drainage device of any example herein, particularly example 1, wherein the porous plug is made of a biocompatible material selected from a ceramic, glass, metal, polymer, and combinations thereof.

Example 9. The sterile drainage device of any example herein, particularly to any one of examples 1 to 8, wherein the porous plug further comprises a plug lumen surrounded by the plug inner uncovered surface, wherein the plug lumen is continuous with, and is in fluid communication with, the tube lumen.

Example 10. The sterile drainage device of any example herein, particularly any one of examples 1 to 9, wherein the pores define a mean pore size which is from 1 to 60 μm.

Example 11. The sterile drainage device of any example herein, particularly example 10, wherein the mean pore size is from 1 to 20 μm.

Example 12. The sterile drainage device of any example herein, particularly example 10, wherein the mean pore size is from 3 to 12 μm.

Example 13. The sterile drainage device of any example herein, particularly example 10, wherein the mean pore size is from 4 to 8 μm.

Example 14. The sterile drainage device of any example herein, particularly any one of examples 1 to 13, wherein a plug has a plug total length which is from 1 to 100 mm.

Example 15. The sterile drainage device of any example herein, particularly example 14, wherein the plug total length is at least two times as great as the plug outer dimension.

Example 16. The sterile drainage device of any example herein, particularly example 14, wherein the plug total length is at least three times as great as the plug outer dimension.

Example 17. The sterile drainage device of any example herein, particularly any one of examples 1 to 16, wherein the plug distal portion has an atraumatic shape, devoid of sharp edges.

Example 18. The sterile drainage device of any example herein, particularly any one of examples 1 to 11, wherein the tube distal portion is disposed around the plug proximal portion.

Example 19. The sterile drainage device of any example herein, particularly example 18, wherein the plug proximal portion comprises a plug outer step, such that an outer surface of the drainage tube is flush with the plug exposed outer surface.

Example 20. The sterile drainage device of any example herein, particularly any one of examples 1 to 17, wherein the tube distal portion extends into the plug proximal portion through a plug proximal opening defined at a plug proximal end of the porous plug.

Example 21. The sterile drainage device of any example herein, particularly example 20, wherein the plug proximal portion further comprises a plug inner step, such that an inner surface of the drainage tube is flush with the plug inner uncovered surface.

Example 22. The sterile drainage device of any example herein, particularly any one of examples 1 to 17, wherein the tube distal portion is connected to the plug proximal portion by a hollow connector.

Example 23. The sterile drainage device of any example herein, particularly example 22, wherein the hollow connector comprises a distal extension having an outer threading engaged with an inner threading of the plug lumen, and a proximal extension having one or more tapered barbs.

Example 24. The sterile drainage device of any example herein, particularly example 22 or example 23, further comprising a sealing member disposed between the tube distal portion and the plug proximal portion.

Example 25. The sterile drainage device of any example herein, particularly any one of examples 1 to 24, wherein the porous plug has a circular cross-sectional shape, and wherein the plug outer dimension is a plug outer diameter.

Example 26. The sterile drainage device of any example herein, particularly example 16, wherein the porous plug is cup-shaped.

Example 27. The sterile drainage device of any example herein, particularly example 25 or 26, wherein the porous plug tapers to a narrower plug outer diameter in the distal direction.

Example 28. The sterile drainage device according to any one of claims 1 to 24, wherein the porous plug comprises a first surface extending between two lateral sides of the porous plug, and a second surface extending between the two lateral sides, wherein the first surface is concave relative to the central longitudinal axis, wherein the second surface is convex relative to the central longitudinal axis, wherein the porous plug defines a plug width between the lateral sides, and a plug height between the first surface and the second surface.

Example 29. The sterile drainage device of any example herein, particularly example 28, wherein the first surface and the second surface converge towards each other at the lateral sides.

Example 30. The sterile drainage device of any example herein, particularly any one of examples 28 or 29, wherein the first surface and the second surface have different radii of curvature.

Example 31. The sterile drainage device of any example herein, particularly any one of examples 28 to 30, wherein a maximal value of the plug width is from 5 to 30 mm.

Example 32. The sterile drainage device of any example herein, particularly any one of examples 28 or 31, wherein the plug width is decreased in size in the distal direction.

Example 33. The sterile drainage device of any example herein, particularly any one of examples 28 to 32, wherein a maximal value of the plug height is from 2 to 15 mm.

Example 34. The sterile drainage device of any example herein, particularly any one of examples 28 to 33, wherein the plug height is decreased in size in the distal direction.

Example 35. The sterile drainage device of any example herein, particularly any one of examples 28 to 30, wherein a maximal value of the plug width is at least two times as great as a maximal value of the plug height.

Example 36. The sterile drainage device of any example herein, particularly any one of examples 28 to 30, wherein a maximal value of the plug width is at least three times as great as a maximal value of the plug height.

Example 37. The sterile drainage device of any example herein, particularly any one of examples 1 or 36, wherein the drainage tube comprises an implantable tube portion comprising the tube distal portion, and an extracorporeal tube portion configured to extend outside a patient body when the implantable tube portion is implanted in a patient's body.

Example 38. The sterile drainage device of any example herein, particularly example 37, wherein the extracorporeal tube portion comprises a connector configured to connect with an external pump.

Example 39. The sterile drainage device of any example herein, particularly any one of examples 1 or 36, further comprising an access port attached to a proximal portion of the drainage tube, the access port comprising a housing defining a chamber which is in fluid communication with the tube lumen, and a self-sealing septum attached to the housing and enclosing the chamber.

Example 40. The sterile drainage device of any example herein, particularly example 39, wherein the self-sealing septum comprises rubber or silicone.

Example 41. The sterile drainage device of any example herein, particularly any one of examples 1 or 36, further comprising an implantable pump attached to a proximal portion of the drainage tube, and an implantable outlet tube attached to the implantable pump, the implantable outlet tube extending from the implantable pump to an outlet opening of the implantable outlet tube.

Example 42. The sterile drainage device of any example herein, particularly example 41, wherein the implantable pump comprises a pump inlet port to which the drainage tube is attached, and a pump outlet port to which the implantable outlet tube is attached.

Example 43. The sterile drainage of any example herein, particularly any one of examples 1 to 42, wherein the bodily fluid is selected from lymph fluid, interstitial fluid, and combination thereof.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

What is claimed is:

1. A sterile drainage device for draining bodily fluids, comprising:
a drainage tube comprising a tube distal portion, the drainage tube defining a tube lumen extending along a central longitudinal axis; and
a porous plug comprising:
a plug proximal portion attached to the tube distal portion;
a plug exposed outer surface facing away from the central longitudinal axis and defining a plug outer dimension;
a plug inner uncovered surface which is exposed to, and is in fluid communication with, the tube lumen;
a plurality of pores comprising:
a plurality of pore outer openings at the plug exposed outer surface;
a plurality of pore inner openings at the plug inner uncovered surface; and
a plurality of pore interconnected channels extending through the porous plug, between the pore outer openings and the pore inner openings, such that the pore outer openings are in fluid communication, via the pore interconnected channels and the pore inner openings, with the tube lumen;
wherein a porous rectangular cuboid sample of the porous plug is not bendable to more than 10° when a bending force of 10N or more is applied to a second end thereof while a first end thereof is affixed in position,
wherein the sample is made from the material of the porous plug, comprises a plurality of pores as in the porous plug, has a length×width×height of 50×10× 6 mm, and comprises a lumen extending from the first end along a length of 45 mm, the lumen having a diameter of 1.5 mm;
wherein the porous rectangular cuboid sample is not compressible by more than 10% when a compressive force of 10N or more is applied to a cross-section thereof; and
wherein the porous plug is close-ended at a plug distal portion thereof, such that fluid communication between the tube lumen and an environment surrounding the porous plug is achieved solely through the plurality of pores.

2. The sterile drainage device according to claim 1, wherein the porous plug is made of a biocompatible material selected from a ceramic, glass, metal, polymer, and combinations thereof.

3. The sterile drainage device according to claim 1, wherein the porous plug further comprises a plug lumen surrounded by the plug inner uncovered surface, wherein the plug lumen is continuous with, and is in fluid communication with, the tube lumen.

4. The sterile drainage device according to claim 1, wherein the pores define a mean pore size which is from 1 to 60 μm.

5. The sterile drainage device according to claim 1, wherein a plug has a plug total length which is from 1 to 100 mm.

6. The sterile drainage device according to claim 5, wherein the plug total length is at least two times as great as the plug outer dimension.

7. The sterile drainage device according to claim 1, wherein the tube distal portion is connected to the plug proximal portion by a hollow connector.

8. The sterile drainage device according to claim 7, further comprising a sealing member disposed between the tube distal portion and the plug proximal portion.

9. The sterile drainage device according to claim 1, wherein the porous plug has a circular cross-sectional shape, and wherein the plug outer dimension is a plug outer diameter.

10. The sterile drainage device according to claim 8, wherein the porous plug is cup-shaped.

11. The sterile drainage device according to claim 1, wherein the porous plug comprises a first surface extending between two lateral sides of the porous plug, and a second surface extending between the two lateral sides, wherein the first surface is concave relative to the central longitudinal axis, wherein the second surface is convex relative to the central longitudinal axis, wherein the porous plug defines a plug width between the lateral sides, and a plug height between the first surface and the second surface.

12. The sterile drainage device according to claim 11, wherein the first surface and the second surface have different radii of curvature.

13. The sterile drainage device according to claim 11, wherein the plug width is decreased in size in the distal direction.

14. The sterile drainage device according to claim 11, wherein the plug height is decreased in size in the distal direction.

15. The sterile drainage device according to claim 11, wherein a maximal value of the plug width is at least two times as great as a maximal value of the plug height.

16. The sterile drainage device according to claim 1, wherein the drainage tube comprises an implantable tube portion comprising the tube distal portion, and an extracorporeal tube portion configured to extend outside a patient body when the implantable tube portion is implanted in a patient's body.

17. The sterile drainage device according to claim 16, wherein the extracorporeal tube portion comprises a connector configured to connect with an external pump.

18. The sterile drainage device according to claim 1, further comprising an access port attached to a proximal portion of the drainage tube, the access port comprising a housing defining a chamber which is in fluid communication with the tube lumen, and a self-sealing septum attached to the housing and enclosing the chamber.

19. The sterile drainage device according to claim 18, wherein the self-sealing septum comprises rubber or silicone.

20. The sterile drainage device according to claim 1, further comprising an implantable pump attached to a proximal portion of the drainage tube, and an implantable outlet tube attached to the implantable pump, the implantable outlet tube extending from the implantable pump to an outlet opening of the implantable outlet tube.

21. The sterile drainage device according to claim 20, wherein the implantable pump comprises a pump inlet port to which the drainage tube is attached, and a pump outlet port to which the implantable outlet tube is attached.
